(19) United States
(12) Patent Application Publication
(10) Pub. No.: US 2021/0031005 A1
(43) Pub. Date: Feb. 4, 2021

(54) AN APPLICATOR FOR A URINARY CATHETER

(71) Applicant: Ahmed Rabee Salem Rabei Alhammadi, Abu Dhabi (AE)

(72) Inventor: Ahmed Rabee Salem Rabei Alhammadi, Abu Dhabi (AE)

(21) Appl. No.: 16/967,682

(22) PCT Filed: Feb. 5, 2018
(86) PCT No.: PCT/IB2018/050697

§ 371 Date: Aug. 5, 2020

(51) Int. Cl. A61M 25/01; A61F 5/453; A61M 25/00

(52) U.S. Cl. CPC A61M 25/0111; A61F 5/453; A61M 2205/0216; A61M 2205/0205; A61M 25/0017

(57) ABSTRACT

The invention teaches an applicator (30) for use in catheterizing a male patient to drain fluid from a bladder, the applicator comprises a sleeve (32) for receiving and directing the catheter tube (14) to be passed through the urethra of the user, and a sheath (52) for shielding the urethral meatus and penis of the user during catheterization from potential infectious agents or bacteria which may contact the catheter prior to insertion and be transmitted with the catheter to the bladder, causing bladder infections and the like. The invention further provides a method of catheterization using the aforementioned applicator.

### FIELD OF THE INVENTION

The invention relates to urinary catheters and more particularly to a device for introducing an intermittent male catheter.

### BACKGROUND OF THE INVENTION

Urinary catheters used for draining a user's bladder are commonly known, and include either internal catheters or external catheters. Internal catheters, broadly speaking, are concerned with passing the catheter through the urethra and into the bladder. Catheters carry an inherent risk of transmitting an infection to the bladder due to the nature of its functioning.

When inserting a catheter the area around the urethra is thoroughly cleaned. The catheter is commonly housed in a sterile container before insertion, and the operator is ideally wearing surgical gloves. This is one way to reduce and attempt to eliminate the infections sustained. This may be the instance when inserting a catheter in a hospital, however there are scenarios where catheterization takes place outside of a hospital.

Typically, immobile patients, such as those who have suffered severe spinal trauma, are required to catheterize themselves, sometimes several times a day. Bladder infections as a result of this catheterization are far more prevalent and can be something of a problem for these patients. The most common source of the infections being the contamination of the catheter as it is passed into the bladder.

Many catheters have been developed to address these issues, including providing an applicator associated with the catheter, such as the kind taught in U.S. Pat. No. 6,578,709 (Kavanagh). By inserting a part of the housing into the urethra of the user, the exposure of the catheter to the surroundings is limited. This approach is thought to limit the risk of infection. However, the invention does not address scenarios in which the discomfort or displeasure of having to insert a portion of the housing into the urethra is considered.

External catheters which do not require insertion of the catheter into the bladder provide an alternative to the invasive self-catheterization. Such catheters, commonly designed for use by males, include a sheath adapted to fit over a user's penis, which has a first drainage end and an opposed attachment end. The drainage end includes an opening at the tip to which a drainage tube is attached. While such catheters avoid the intrusive nature of the internal catheters, they are not without their own drawbacks, including the risk of leakage.

### OBJECT OF THE INVENTION

It is an object of the current invention to address the above mentioned problems, at least to a degree, by providing a male catheter which limits the risk of leakage and infection whilst avoiding the discomfort and displeasure of unnecessary urethral disruption.

### SUMMARY OF THE INVENTION

In accordance with the invention there is provided an applicator for a urinary catheter which includes;
a sleeve comprising a leading portion and an opposed trailing portion and a passage which extends therethrough, the passage being open at each end; and
a sheath of stretchable, elastomeric material having a first end, secured about the sleeve, and an opposed free end, wherein;
the sheath is extendable from the first end to engage the penis of a user so as to create an enclosed area between the sleeve and the penis.

The invention provides for the sheath to be rolled upon itself before use; further for the sheath to be unrolled coaxially along the leading portion when in use; for the sheath to be unrolled from the first end towards the opposed free end; further for the sheath, when unrolled, to extend past the leading portion.

The invention provides for the sheath to be substantially tubular in shape; further for the tubular sheath to include an inner surface and an outer surface and for the inner surface to come into contact with the glans when in use; further for the inner surface to be coated in an anti-bacterial composition; further still for the inner surface to be coated in an adhesive composition.

The invention provides for the sleeve to include a collar positioned between the leading portion and trailing portion; further for the collar to define a base of the leading portion at which the leading portion and collar meet; further still for the secured end of the sheath to be located at said base.

The invention provides for the sleeve to be substantially cylindrical in shape, wherein the leading and trailing portions are similarly cylindrical; further for passage to be cylindrical and include a diameter slightly larger than the outer diameter of a catheter.

The invention provides further for the leading portion to include a seal at the opening; further for the seal to restrain movement of a catheter passed through the passage; further still for the seal to allow movement of a catheter passed through the passage when an axial force is applied to overcome the threshold resistance of the seal.

The invention provides for the trailing end to include a plurality of gripping formations; further where the gripping formations may be a set of ribs and/or projections, wherein the formations provide an undulating surface for securing to a conduit.

The invention provides further still for the applicator to include a protective cap; further where the cap attaches to an outer periphery of the collar so as to cover the leading portion and sheath prior to use of the device.

### BRIEF DESCRIPTION OF THE DRAWINGS

These and other features of the invention will become more apparent by the following description of the embodiment, which is made by way of example, with reference to the accompanying drawings in which:

FIG. 1: shows a view, in perspective, of an internal male catheter kit common within the art;

FIG. 2: shows a view in perspective of an Applicator in accordance with the invention;

FIG. 3: shows a view in perspective of the applicator of FIG. 2, with the sheath unrolled; and

FIG. 4: shows perspective views, in a stepwise fashion, of the catheter in use in accordance with the invention.

BRIEF DESCRIPTION OF THE PREFERRED EMBODIMENTS

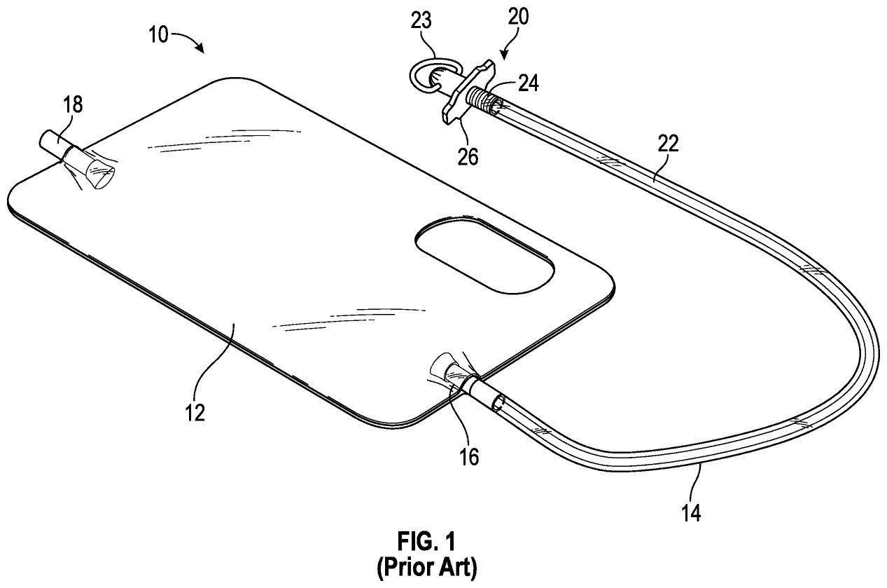

Referring to FIG. 1, which shows a common external male urinary catheter assembly 10, available in the art. While certain components may differ or vary in shape and size the general components bear a similar resemblance and function to those shown in the figure.

The components which make up the assembly 10 include; a collection (or discharge) bag 12, a catheter tube 14, a catheter funnel 16, a drain valve 18, guide member 20, catheter 22 and ring cap 23.

Collection bag 12 includes two openings. The first opening includes the drain valve 18 for emptying the bag as and when necessary. The second opening includes the catheter funnel 16. The funnel connects, and places, the collection bag and catheter 22 in fluid communication.

The catheter tube 14 is connected, at one end, to the collection bag 12 through the collection funnel 14, and at the other end, to the guide member 20.

Positioned within the catheter tube and extending from the collection bag is the catheter 22. The insertion end of the catheter may include one or more eyelets (not shown) for allowing passage of fluids into the catheter.

The guide member 20 includes a leading end (obscured in the figure) and a trailing end 24, separated by an outwardly projecting collar 26. A closure arrangement, (also not shown) for retaining the catheter 22 when not in use, forms part of the leading end. A ring cap 23 is provided which engages with the collar and covers the leading end and closure arrangement of the guide member.

In use, ring cap 23 is removed from the guide member 20 to expose the leading end. The leading end is inserted into the urethral meatus of a user with the insertion end of the catheter being safely retained behind the closure arrangement. Once the guide member is in position the catheter is slid toward the closure arrangement with sufficient force to overcome the threshold resistance of the arrangement. The catheter then moves into the urethra and is continually driven until the insertion end enters the user's bladder.

Once in this position any fluid to be drained will enter the catheter through the eyelets and flow through the catheter until passing the catheter funnel and ending in the collection bag 12. Once the bladder is sufficiently drained the catheter may be withdrawn, the associated components removed and the collection bag emptied, or discarded.

With the above described assembly and method of use there exist occasions in which infections are passed from the catheter into the bladder of the user. One scenario is that the catheter will collect bacteria in the non-sterile area of the urethra, which is just before the high pressure, sterile, area of the urethra, and carry it through to the bladder, where it may eventually cause the infection.

The use of the leading end of the guide member is thought to limit this type of infection by not allowing the catheter to pass through the non-sterile portion of the urethra. The leading end is inserted into the non-sterile portion and possibly just past. Thereafter the catheter is passed into the urethra without contacting the non-sterile portion, therefore, hopefully reducing the risk of bacteria being introduced to the bladder.

However, notwithstanding the above, infections still occur. The belief is that the surrounding area between the guide member, glans and meatus urethra may still play a role in causing the infections. The object of the current invention seeks to limit the exposure of the user and the components of a catheter assembly to this potentially infectious causing area. Further, the increased diameter of the leading portion has, in certain instances, been found to be quite disruptive to the urethra of a user, causing a degree of displeasure and discomfort. The abovementioned approach has failed to account for such instances, the limitation of which is a further objective of the current invention.

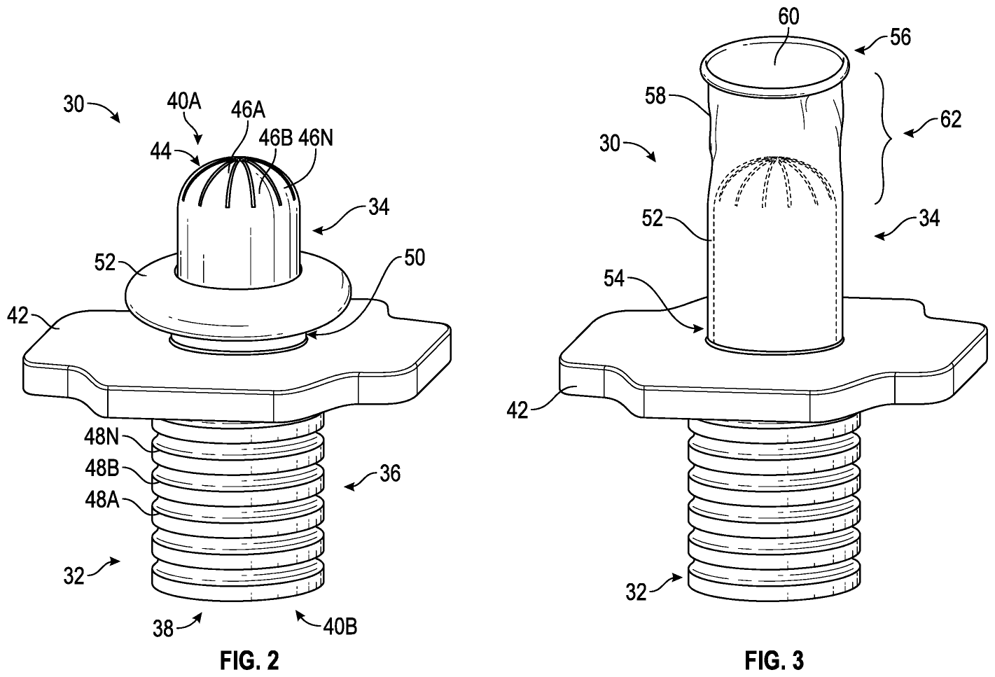

Turning to FIG. 2 which shows an Applicator 30 for a urinary catheter, in accordance with the invention.

The applicator 30 is in the form of sleeve 32. The sleeve includes a leading portion 34, a trailing portion 36 and an internal passageway 38 which passes therethrough. A pair of openings 40A and 40B flank each end of the passageway. The sleeve additionally includes an outwardly projecting collar 42 positioned between the leading portion and the trailing portion.

The sleeve 32 is substantially cylindrical in shape, as is internal passageway 38. The passageway has a substantially uniform diameter along its length. The diameter is chosen to allow a catheter to pass therethrough with a degree of play.

Located at the opening 40A of the leading portion 34 is sealing arrangement 44. The arrangement is formed as of a plurality of resiliently deformable flaps 46A, 46B . . . 46N which taper inwardly from the leading portion to block the opening. A force applied against the seal by an object, which overcomes the threshold resistance of the seal, will cause the flaps to divert and unblock the opening. The flaps will return to blocking the opening should the object be removed.

The external surface of the trailing portion 36 includes a plurality of gripping formations in the form of encircling ribs 48A, 48B . . . 48N. The ribs provide an undulating surface for securing the sleeve to an inner surface of a conduit or pipe. The ribs extend so far as the collar 42.

Leading portion 34 meets the collar 42 at a base region 50.

The sleeve 12 additionally includes sheath 52. The sheath is formed of an elastomeric, stretchable material and is secured to the base region 50, at a first end 54. In the figure the sheath is rolled on itself, which makes viewing the leading portion 34 possible.

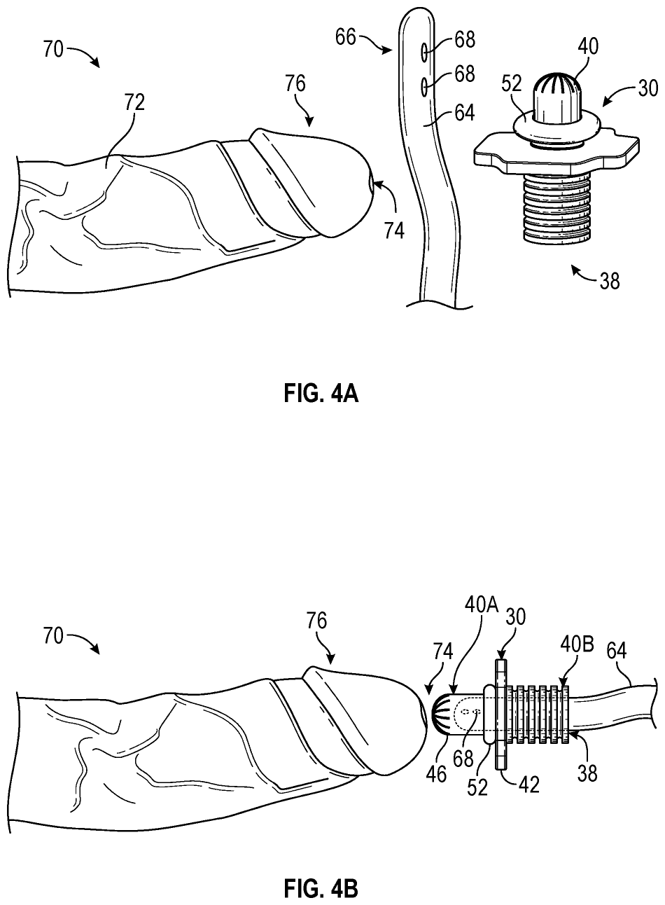

Turning to FIG. 3, the sheath 52 is unrolled and partially obscures viewing the leading portion 34. The sheath is substantially tubular in shape and extends from the secured first end 54 to an opposing free end 56. The sheath includes an outer surface 58 and an inner surface 60.

Importantly, and as shown in FIG. 3, when the sheath 52 is in an unrolled state the free end 56 extends past the opening 40A of the leading portion 34. This extension piece, referred to as the capture section 62, acts to receive part of a user's penis when in use, as discussed in greater detail below.

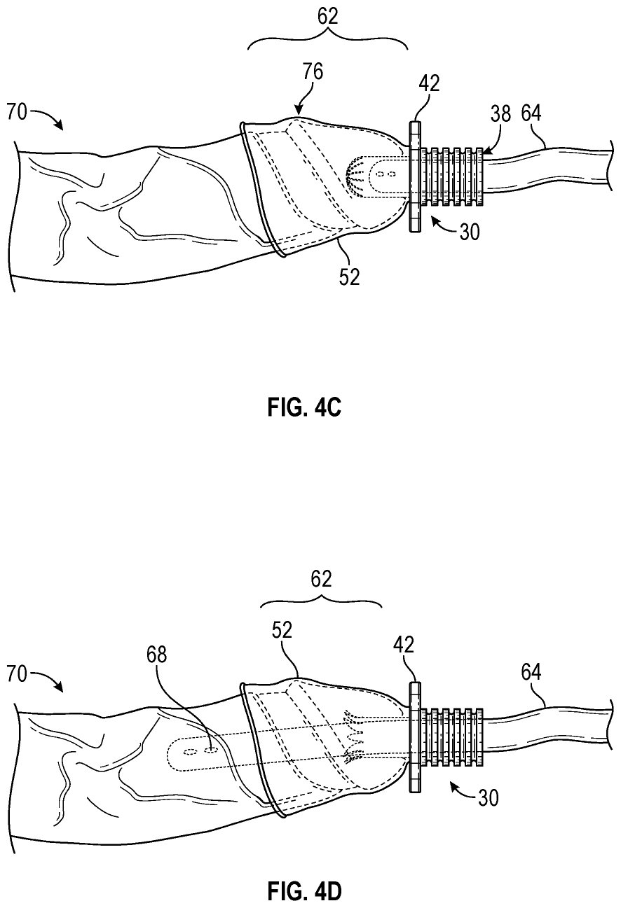

Turning to FIG. 4 which shows a stepwise approach to using the invention.

FIG. 4A depicts the applicator 30, a catheter 64, having an insertion end 66 and eyelets 68 positioned toward the insertion end, for providing passage of fluids into the catheter, and a body 70 which is to be catheterized. The body includes the male penis 72, depicting the urethral meatus 74 and glans 76.

In FIG. 4B the catheter 64 is received within passageway 38 by longitudinally passing the insertion end 66 through opening 40B. The catheter is continually fed until the insertion end abuts the sealing arrangement 44. The catheter is thus retained behind flaps 46.

The applicator 30 is aligned, with opening 40A of the leading end 34 directed toward the urethral meatus 74 of a user, while the sheath 52 is in a rolled state.

FIG. 4C shows the next step in the process. The leading portion 34 has been pressed, to a degree, into the urethral meatus 74, until the collar abuts the tip of the glans 76. Additionally, the sheath 52 has been unrolled from the secured end 54, coaxially with the leading portion, so as to receive at least the glans within the free end 56. The glans is held within capture section 62.

The surrounding area between the urethral meatus, glans and leading portion of the applicator is now shielded from bacteria or infectious agents by capture section 62. The inner surface 60 may be coated with antibacterial compositions to create sterile conditions, and may also be coated with an adhesive composition for better securing to the glans 76, and any additional portion of the penis 72.

FIG. 4D shows the last step where the catheter is fed into, and through, the urethra. By applying a longitudinal force to the catheter sufficient to overcome the threshold resistance of flaps 46, the insertion end 66 moves into the user's urethra. The catheter is continually fed until the bladder is reached. Any fluids to be drained flow through eyelets 68, through the catheter and out of the system, commonly into a collection or drainage bag.

Further to the invention, there is an embodiment of use wherein the displeasure and discomfort of pressing the leading end 34 into the urethral meatus is avoided.

In this embodiment, the applicator 30 is aligned in much the same fashion as that described in FIG. 4B. In terms of a next step however, i.e. as described in FIG. 4C, the leading end 34 in not pressed further than merely placing the flaps 46 in contact with the urethral meatus. The reaming steps of unrolling the sheath 52 to receive the at least the glans 76, and holding the glans within the capture section 62 are, however, retained.

A longitudinal force sufficient to overcome the resistance of flaps 46 is applied such that insertion end 66 is moved into the user's urethra, and the remaining process remains. The difference residing that the applicator is held in the aligned position until the catheterization process has been completed, and the catheter removed.

A person skilled in the art will appreciate that a number of variations may be made to the above described invention or features thereof, without departing from the scope of the present invention.

1. An applicator for an external male urinary catheter comprising:
a sleeve comprising a leading portion, a trailing portion and a passage, shaped to receive a catheter passed therethrough, which extends through the sleeve, the passage being open at each end;
the leading portion comprising a seal at the opening to restrain movement of a catheter passed therethrough;
the seal being openable upon a threshold resistance of the seal being overcome by a catheter being pressed therethrough, wherein;
the leading portion is inserted into a urethral meatus of a user prior to the catheter being pressed through the seal; and
a sheath of stretchable, elastomeric material having a first end, secured about the sleeve, and an opposed free end, wherein;
the sheath is extendable from the first end to engage the penis of a user so as to create an enclosed area between the sleeve and the penis.

2. An applicator as claimed in claim 1 wherein the sheath includes an inner surface and an outer surface.

3. An applicator as claimed in claim 2 wherein the inner surface includes at least an anti-bacterial composition and at least an adhesive composition.

4. An applicator as claimed in claim 3 wherein the sheath is tubular in shape.

5. An applicator as claimed in claim 1 wherein the sleeve includes an outwardly extending collar positioned between the leading portion and the trailing portion.

6. An applicator as claimed in claim 5 further comprising a base located between the leading portion and the collar, wherein the first end of the sheath is secured to the base.

7. An applicator as claimed in claim 5 further comprising a protective cap, wherein the cap attaches to an outer periphery of the collar so as to cover the leading portion and sheath.

8-10. (canceled)

11. An applicator as claimed in claim 1 further comprising a plurality of gripping formations positioned along the trailing portion.

12-13. (canceled)

14. An applicator as claimed in claim 4 wherein the sleeve includes an outwardly extending collar positioned between the leading portion and the trailing portion.

15. An applicator as claimed in claim 14 further comprising a base located between the leading portion and the collar, wherein the first end of the sheath is secured to the base.

16. An applicator as claimed in claim 15 further comprising a protective cap, wherein the cap attaches to an outer periphery of the collar so as to cover the leading portion and sheath.

17. A method for using an applicator for urinary catheterization of a male patient comprising:
providing an applicator having a sleeve comprising a leading portion, a trailing portion and a passage which extends therethrough, the passage being open at each end; and a sheath of stretchable, elastomeric material having a first end, secured about the sleeve, and an opposed free end;
inserting the leading portion of the sleeve into the urethra;
extending the sheath such that the free end extends away from the secured first end;
passing the free end over the penis of the patient so as to create an enclosed area between the penis and the sleeve;
pressing the catheter through the passage of the sleeve such that the catheter presses against a seal at the opening of the leading portion;
applying a force to the catheter such that the threshold resistance of the seal is overcome so that the catheter is passed into the urethra of the patient; and
continuing to pass the catheter through the urethra until reaching the bladder.

18. A method for using an applicator for urinary catherization of a male patient comprising:
providing an applicator having a sleeve comprising a leading portion, a trailing portion and a passage which extends therethrough, the passage being open at each end; and a sheath of stretchable, elastomeric material having a first end, secured about the sleeve, and an opposed free end;
aligning the opening of the leading portion of the sleeve with the urethral meatus of the patient;
extending the sheath such that the free end extends away from the secured first end;
passing the free end over the penis of the patient so as to create an enclosed area between the penis and the sleeve;
pressing the catheter through the passage of the sleeve such that the catheter presses against a seal at the opening of the leading portion;
applying a force to the catheter such that the threshold resistance of the seal is overcome so that the catheter is exposed to the enclosed area between the penis and the sleeve;
passing the catheter through the urethral meatus and into the urethra of the patient; and
continuing to pass the catheter through the urethra until reaching the bladder.
